(19) United States
(12) Patent Application Publication
(10) Pub. No.: US 2021/0313476 A1
(43) Pub. Date: Oct. 7, 2021

(54) OPTICAL SENSOR PACKAGE STRUCTURE AND OPTICAL MODULE STRUCTURE

(71) Applicant: Advanced Semiconductor Engineering, Inc., Kaohsiung (TW)

(72) Inventors: Chun Yu KO, Kaohsiung (TW); Tsu-Hsiu WU, Kaohsiung (TW); Wei-Tang CHU, Kaohsiung (TW)

(73) Assignee: Advanced Semiconductor Engineering, Inc., Kaohsiung (TW)

(21) Appl. No.: 16/838,677

(22) Filed: Apr. 2, 2020

(51) Int. Cl. H01L 31/0203; H01L 31/02; H01L 31/0232; H01L 31/167; H01L 31/18

(52) U.S. Cl. CPC H01L 31/0203; H01L 31/02005; A61B 5/14552; H01L 31/167; H01L 31/18; H01L 31/02325

(57) ABSTRACT

An optical sensor package structure and an optical module structure are provided. The optical sensor package structure includes a substrate, a sensor device and a transparent encapsulant. The sensor device is electrically connected to the substrate, and has a sensing area facing the substrate. The transparent encapsulant covers the sensing area of the sensor device.

### BACKGROUND

### 1. Field of the Disclosure

The present disclosure relates to an optical sensor package structure and an optical module structure, and to an optical sensor package structure including a transparent encapsulant, and an optical module structure including the same.

### 2. Description of the Related Art

Optical sensor devices are widely used in health monitors to determine physiological characteristics of a person because of a non-invasive nature. For example, a health monitor having an optical sensor device, e.g., an oxihemometer, is a non-invasive apparatus for monitoring a person's blood oxygen saturation. An optical sensor device may be placed on a thin part of the person's body, usually a fingertip or earlobe, or in the case of an infant, across a foot. The optical sensor device passes two wavelengths of light through the body part to a photodetector. The changing absorbance at each of the wavelengths is measured, allowing the health monitor to determine the absorbance of the pulsing blood.

### SUMMARY

In some embodiments, an optical sensor package structure includes a substrate, a sensor device and a transparent encapsulant. The sensor device is electrically connected to the substrate, and has a sensing area facing the substrate. The transparent encapsulant covers the sensing area of the sensor device.

In some embodiments, an optical sensor package structure includes a transparent substrate, a sensor device and a transparent encapsulant. The sensor device is electrically connected to the transparent substrate, and has a sensing area facing the transparent substrate. The transparent encapsulant covers the sensor device and a surface of the transparent substrate. A ratio of a refractive index of the transparent encapsulant to a refractive index of the transparent substrate is in a range of 0.98 to 1.02.

In some embodiments, an optical module structure includes a substrate, a light transmitter, a light receiver and a first encapsulant. The light transmitter is attached to the substrate. The light receiver is attached to the substrate and has a sensing area. The first encapsulant covers the light receiver and a first portion of the substrate. The first encapsulant is transparent and covers the sensing area of the light receiver.

### BRIEF DESCRIPTION OF THE DRAWINGS

Aspects of some embodiments of the present disclosure are readily understood from the following detailed description when read with the accompanying figures. It is noted that various structures may not be drawn to scale, and dimensions of the various structures may be arbitrarily increased or reduced for clarity of discussion.

FIG. 1 illustrates a cross-sectional of an optical sensor package structure according to some embodiments of the present disclosure.

FIG. 2 illustrates a top perspective view of the optical sensor package structure of FIG. 1.

FIG. 3 illustrates a bottom perspective view of the optical sensor package structure of FIG. 1.

FIG. 4 illustrates a cross-sectional view taken along line 4-4 of the optical sensor package structure of FIG. 2.

FIG. 5 illustrates a simulation result of a relationship between the optical signal-to-noise ratio (OSNR) of an optical signal and the refractive index of a transparent encapsulant, wherein the optical signal of FIG. 4 has different incident angles.

FIG. 6 illustrates a simulation result of a relationship between the optical signal-to-noise ratio (OSNR) of an optical signal and the refractive index of the transparent encapsulant, wherein the optical signal of FIG. 4 has different incident angles.

FIG. 7 illustrates a simulation result of a relationship between the optical signal-to-noise ratio (OSNR) of an optical signal and the refractive index of the transparent encapsulant, wherein the optical signal of FIG. 4 has different incident angles.

FIG. 8 illustrates a simulation result of a relationship between the optical signal-to-noise ratio (OSNR) of an optical signal and the refractive index of the transparent encapsulant, wherein the optical signal of FIG. 4 has different incident angles.

FIG. 9 illustrates a cross-sectional view of an optical sensor package structure according to some embodiments of the present disclosure.

FIG. 10 illustrates a cross-sectional view of an optical sensor package structure according to some embodiments of the present disclosure.

FIG. 11 illustrates a cross-sectional view of an optical module structure according to some embodiments of the present disclosure.

FIG. 12 illustrates one or more stages of an example of a method for manufacturing an optical sensor package structure according to some embodiments of the present disclosure.

FIG. 13 illustrates one or more stages of an example of a method for manufacturing an optical sensor package structure according to some embodiments of the present disclosure.

FIG. 14 illustrates one or more stages of an example of a method for manufacturing an optical sensor package structure according to some embodiments of the present disclosure.

FIG. 15 illustrates one or more stages of an example of a method for manufacturing an optical sensor package structure according to some embodiments of the present disclosure.

### DETAILED DESCRIPTION

Common reference numerals are used throughout the drawings and the detailed description to indicate the same or similar components. Embodiments of the present disclosure will be readily understood from the following detailed description taken in conjunction with the accompanying drawings.

The following disclosure provides many different embodiments, or examples, for implementing different features of the provided subject matter. Specific examples of components and arrangements are described below to explain certain aspects of the present disclosure. These are, of course, merely examples and are not intended to be limiting. For example, the formation of a first feature over or on a second feature in the description that follows may include embodiments in which the first and second features are formed or disposed in direct contact, and may also include embodiments in which additional features may be formed or disposed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

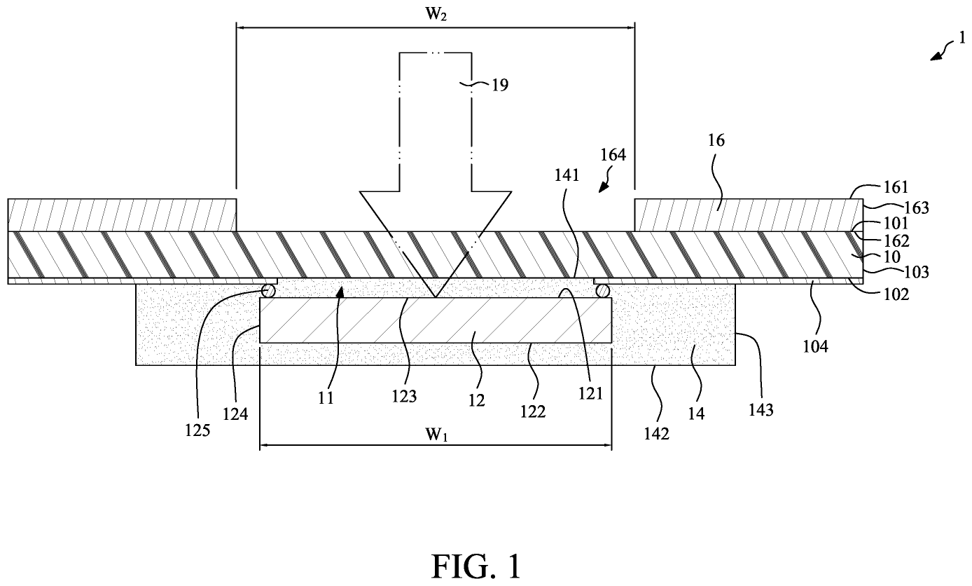

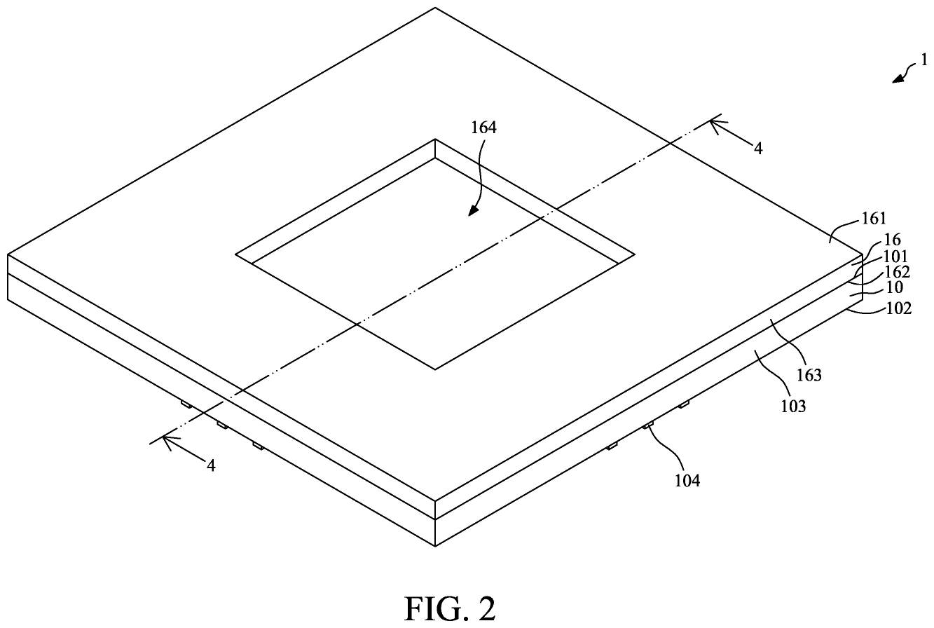

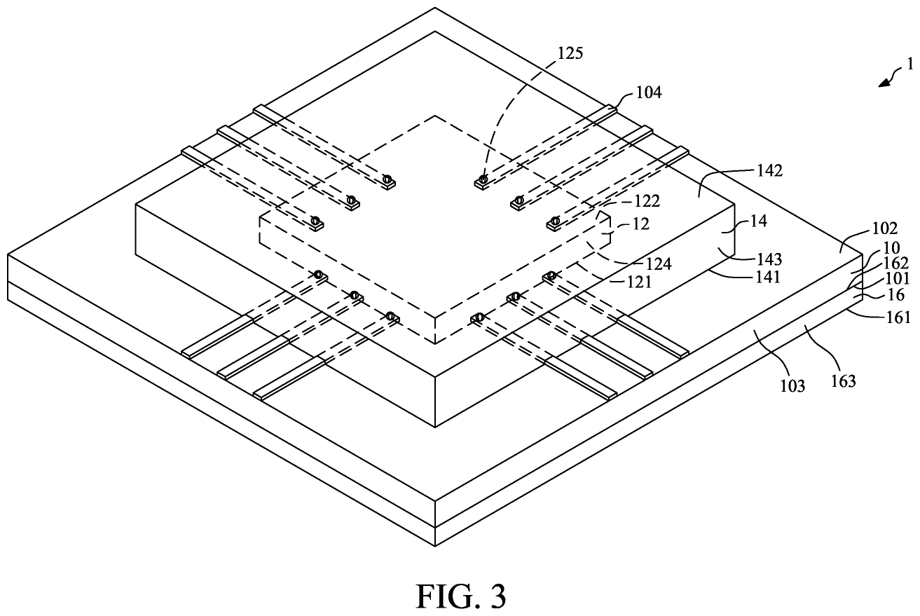

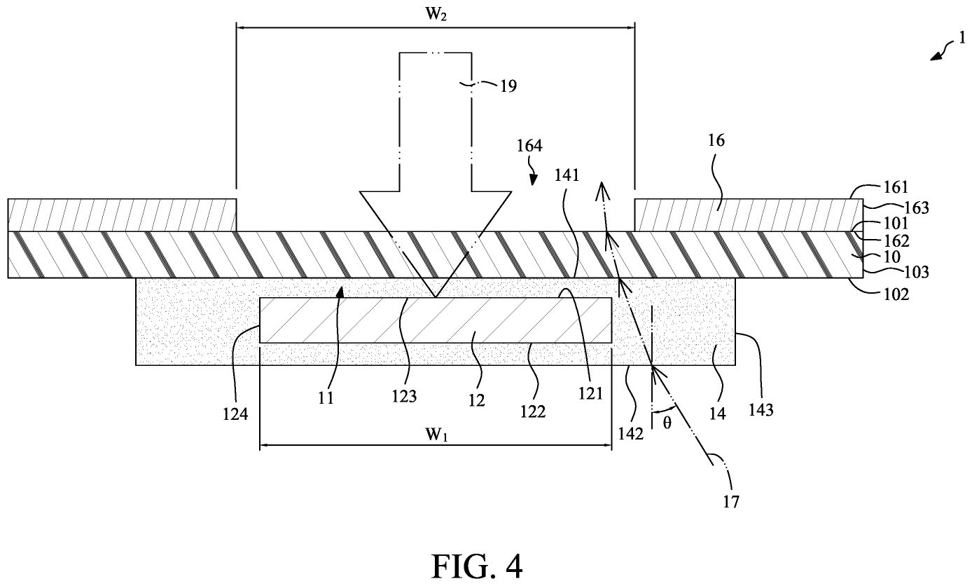

FIG. 1 illustrates a cross-sectional of an optical sensor package structure 1 according to some embodiments of the present disclosure. FIG. 2 illustrates a top perspective view of the optical sensor package structure 1 of FIG. 1. FIG. 3 illustrates a bottom perspective view of the optical sensor package structure 1 of FIG. 1. FIG. 4 illustrates a cross-sectional view taken along line 4-4 of the optical sensor package structure 1 of FIG. 2. The optical sensor package structure 1 includes a substrate 10, a sensor device 12, a transparent encapsulant 14 and a mask layer 16.

The substrate 10 may be transparent. Thus, the substrate 10 may be also referred to as a “transparent substrate”. In some embodiments, a material of the substrate 10 may be transparent, and can be seen through or detected by human eyes or machine (e.g., charge-coupled device (CCD)). In some embodiments, a transparent material of the substrate 10 has a light transmission of at least about 60%, at least about 70%, or at least about 80% for a wavelength in the visible range. The wavelength in the visible range may be in a range of 400 nm to 700 nm. A material of the substrate 10 may include glass. In addition, a refractive index of the substrate 10 may be in a range of about 1.46 to about 1.85.

The substrate 10 may have a first surface 101 (e.g., a top surface), a second surface 102 (e.g., a bottom surface) opposite to the first surface 101, and four lateral side surfaces 103 extending between the first surface 101 and the second surface 102. In some embodiments, the substrate 10 may further include a circuit layer 104 disposed adjacent to or disposed on the second surface 102 of the substrate 10. The circuit layer 104 may include conductive material, for example but is not limited to Cu, Au, Ag, Al, Ti, Indium Tin Oxide (ITO) or another suitable metal or alloy. The circuit layer 104 may include a plurality of traces, a plurality of pads or other conductive connections.

The sensor device 12 may be electrically connected to the substrate 10, and may have a first surface 121 (e.g., an active surface), a second surface 122 (e.g., a backside surface) opposite to the first surface 121, and four lateral side surfaces 124 extending between the first surface 121 and the second surface 122. In addition, the sensor device 12 may further have a sensing area 123 disposed adjacent to the first surface 121. The sensor device 12 may include a sensing circuit disposed in the sensing area 123 for sensing or detecting an optical signal 19 (e.g., a light). As shown in FIG. 1, the sensor device 12 is electrically connected to the substrate 10 through a flip-chip bonding. That is, the first surface 121 of the sensor device 12 is electrically connected to the circuit layer 104 of the substrate 10 through a plurality of bumps 125. Thus, the sensing area 123 of the sensor device 12 faces the substrate 10, and a gap 11 or a space is formed between the first surface 121 (or the sensing area 123) of the sensor device 12 and the second surface 102 of the substrate 10. A height of the gap 11 may be determined by the height of the bump 125.

The transparent encapsulant 14 may be disposed on the second surface 102 of the substrate 10 to cover the sensor device 12 and the second surface 102 of the substrate 10. As shown in FIG. 1, the transparent encapsulant 14 may have a first surface 141 (e.g., a top surface), a second surface 142 (e.g., a bottom surface) opposite to the first surface 141, and four lateral side surfaces 143 extending between the first surface 141 and the second surface 142. The first surface 141 of the transparent encapsulant 14 may contact the second surface 102 of the substrate 10. For example, the transparent encapsulant 14 may include an optical molding compound such as epoxy resin with or without fillers. In some embodiments, a transparent material of the transparent encapsulant 14 has a light transmission of at least about 60%, at least about 70%, or at least about 80% for a wavelength in the visible range. The wavelength in the visible range may be in a range of 400 nm to 700 nm. In addition, a ratio of a refractive index of the transparent encapsulant 14 to a refractive index of the substrate 10 may be in a range of about 0.98 to about 1.02. That is, the refractive index of the transparent encapsulant 14 is substantially equal to the refractive index of the substrate 10 times (1±2%).

As shown in FIG. 1, a portion of the transparent encapsulant 14 fills the gap 11 between the sensor device 12 and the substrate 10. Thus, the gap 11 may be not an empty space, and the transparent encapsulant 14 may cover the sensing area 123 of the sensor device 12. In some embodiments, the transparent encapsulant 14 may further cover the six side surfaces (including the first surface 121, the second surface 122 and the four lateral side surfaces 124) of the sensor device 12. In addition, the transparent encapsulant 14 may further cover the bumps 125 and a portion of the circuit layer 104 of the substrate 10.

The mask layer 16 may be disposed on the first surface 101 of the substrate 10 opposite to the sensor device 12. As shown in FIG. 1, the mask layer 16 may have a first surface 161 (e.g., a top surface), a second surface 162 (e.g., a bottom surface) opposite to the first surface 161, and four lateral side surfaces 163 extending between the first surface 161 and the second surface 162. The first surface 161 of the mask layer 16 may contact the first surface 101 of the substrate 10. For example, the mask layer 16 may be an opaque light-block material, such as a solder mask resin including carbon black or pigment to absorb or reflect the visible light. In some embodiments, the material of the mask layer 16 has a light transmission of less than about 10%, less than about 5%, less than about 1%, or less than about 0.1% for a wavelength in the visible range.

In addition, the mask layer 16 defines an opening 164 corresponding to the sensor device 12. Thus, only the desired optical signal 19 passing through the opening 164 of the mask layer 16 may enter the sensing area 123 of the sensor device 12 through the substrate 10 and the portion of the transparent encapsulant 14 in the gap 11. The optical signal (or light) that does not pass through the opening 164 of the mask layer 16 may be absorbed or reflected by the mask layer 16. Thus, the mask layer 16 can allow specific optical signal (or light) to enter the sensing area 123 of the sensor device 12, and can prevent undesired optical signal (or light) from entering the sensing area 123 of the sensor device 12.

In some embodiments, a size (e.g., a width W2) of the opening 164 of the mask layer 16 may be slightly greater than a size (e.g., a width W0 of the sensor device 12. Thus, some undesired ambient light 17 (FIG. 4) that comes from the second surface 142 of the transparent encapsulant 14 may pass through the opening 164 of the mask layer 16 and emits out of the optical sensor package structure 1. That is, such undesired ambient light 17 (FIG. 4) coming from the second surface 142 of the transparent encapsulant 14 may not be reflected by the second surface 162 of the mask layer 16 to reach the sensing area 123 of the sensor device 12. Such undesired ambient light 17 (FIG. 4) has an incident angle θ.

In the embodiment illustrated in FIG. 1 to FIG. 3, the sensor device 12 is electrically connected to the substrate 10 through a flip-chip bonding, thus, a total thickness of the optical sensor package structure 1 is reduced. Further, the transparent encapsulant 14 is a transparent material, thus, it may enter the gap 11 between the sensor device 12 and the substrate 10, which may reduce the difficulty of the molding process of the transparent encapsulant 14. In addition, the refractive index of the transparent encapsulant 14 is relatively high, thus, if some undesired ambient light 17 that comes from the second surface 142 of the transparent encapsulant 14 reaches the interface (e.g., the second surface 102 of the substrate 10) between the transparent encapsulant 14 and the substrate 10, such undesired ambient light 17 may not be reflected by the substrate 10 to reach the sensing area 123 of the sensor device 12, which may reduce optical cross-talk between such undesired ambient light 17 and the desired optical signal 19 passing through the opening 164 of the mask layer 16. As a result, an optical signal-to-noise ratio (OSNR) of the optical signal 19 received by the sensor device 12 may be greater than 20 db.

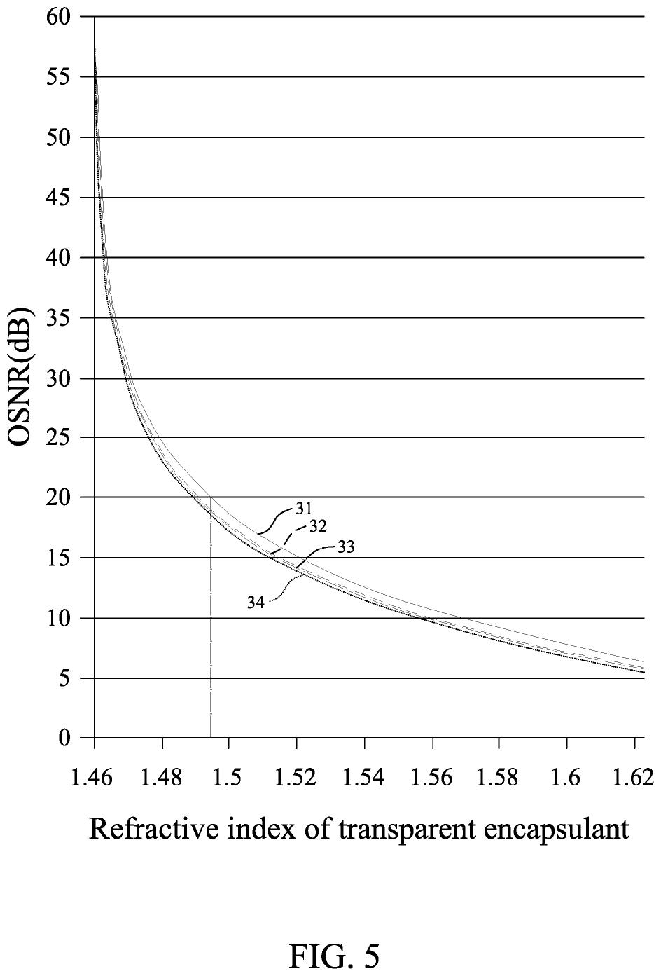

FIG. 5 illustrates a simulation result of a relationship between the optical signal-to-noise ratio (OSNR) of the optical signal 19 and the refractive index of the transparent encapsulant 14 wherein the undesired ambient light 17 of FIG. 4 has different incident angles 0. In FIG. 5, the substrate 10 is predetermined to be a fused silicate glass having a refractive index of 1.46. The curve 31 represents a simulation result when the incident angle θ of FIG. 4 is 60 degrees. The curve 32 represents a simulation result when the incident angle θ of FIG. 4 is 45 degrees. The curve 33 represents a simulation result when the incident angle θ of FIG. 4 is 30 degrees. The curve 34 represents a simulation result when the incident angle θ of FIG. 4 is 15 degrees. As shown in FIG. 5, the curves 31, 32, 33, 34 are substantially consistent with each other. If the target value of the optical signal-to-noise ratio (OSNR) is set to be greater than or equal to 20 dB, the selectable refractive index of the transparent encapsulant 14 may be in a range of about 1.46 to about 1.49. Thus, a ratio of the refractive index of the transparent encapsulant 14 to the refractive index of the substrate 10 may be in a range of about 1.0 to about 1.02.

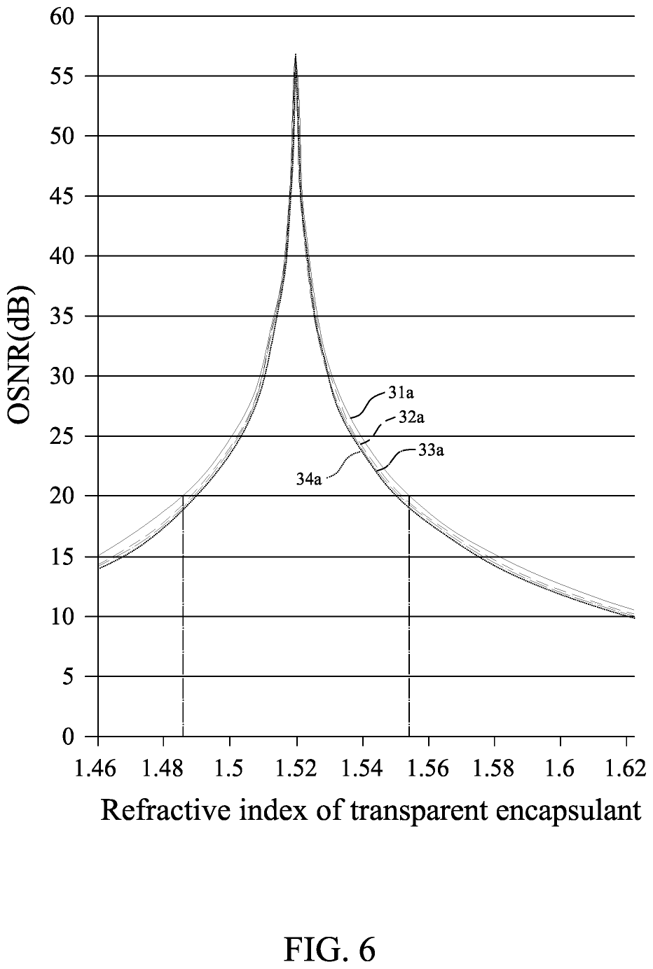

FIG. 6 illustrates a simulation result of a relationship between the optical signal-to-noise ratio (OSNR) of the optical signal 19 and the refractive index of the transparent encapsulant 14 wherein the undesired ambient light 17 of FIG. 4 has different incident angles θ. In FIG. 6, the substrate 10 is predetermined to be a borosilicate glass having a refractive index of 1.52. The curve 31a represents a simulation result when the incident angle θ of FIG. 4 is 60 degrees. The curve 32a represents a simulation result when the incident angle θ of FIG. 4 is 45 degrees. The curve 33a represents a simulation result when the incident angle θ of FIG. 4 is 30 degrees. The curve 34a represents a simulation result when the incident angle θ of FIG. 4 is 15 degrees. As shown in FIG. 6, the curves 31a, 32a, 33a, 34a are substantially consistent with each other. If the target value of the optical signal-to-noise ratio (OSNR) is set to be greater than or equal to 20 dB, the selectable refractive index of the transparent encapsulant 14 may be in a range of about 1.49 to about 1.55. Thus, a ratio of the refractive index of the transparent encapsulant 14 to the refractive index of the substrate 10 may be in a range of about 0.98 to about 1.02.

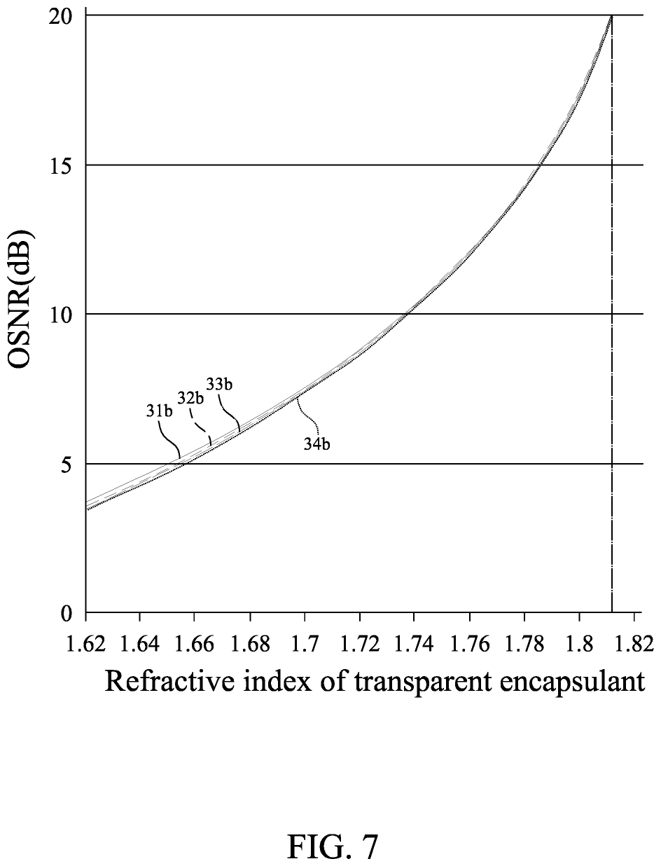

FIG. 7 illustrates a simulation result of a relationship between the optical signal-to-noise ratio (OSNR) of the optical signal 19 and the refractive index of the transparent encapsulant 14 wherein the undesired ambient light 17 of FIG. 4 has different incident angles θ. In FIG. 7, the substrate 10 is predetermined to be a LaSFN9 glass having a refractive index of 1.85. The curve 31b represents a simulation result when the incident angle θ of FIG. 4 is 60 degrees. The curve 32b represents a simulation result when the incident angle θ of FIG. 4 is 45 degrees. The curve 33b represents a simulation result when the incident angle θ of FIG. 4 is 30 degrees. The curve 34b represents a simulation result when the incident angle θ of FIG. 4 is 15 degrees. As shown in FIG. 7, the curves 31b, 32b, 33b, 34b are substantially consistent with each other. If the target value of the optical signal-to-noise ratio (OSNR) is set to be greater than or equal to 20 dB, the selectable refractive index of the transparent encapsulant 14 may be about 1.815. Thus, a ratio of the refractive index of the transparent encapsulant 14 to the refractive index of the substrate 10 may be about 0.98.

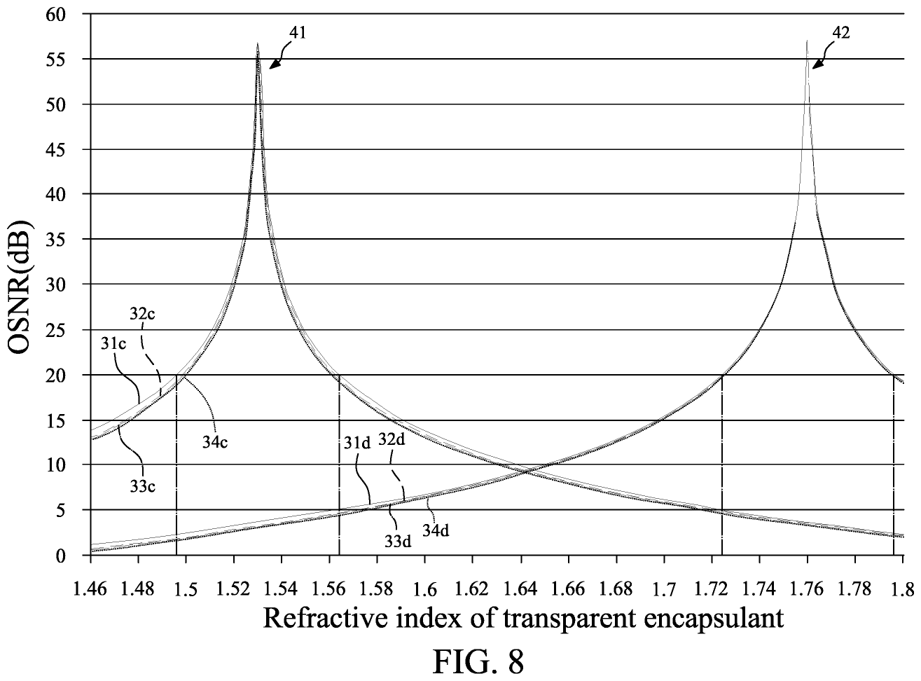

FIG. 8 illustrates a simulation result of a relationship between the optical signal-to-noise ratio (OSNR) of the optical signal 19 and the refractive index of the transparent encapsulant 14 wherein the undesired ambient light 17 of FIG. 4 has different incident angles θ. In the case 41 of FIG. 8, the substrate 10 is predetermined to be an ideal substrate having a refractive index of 1.53. The curve 31c represents a simulation result when the incident angle θ of FIG. 4 is 60 degrees. The curve 32c represents a simulation result when the incident angle θ of FIG. 4 is 45 degrees. The curve 33c represents a simulation result when the incident angle θ of FIG. 4 is 30 degrees. The curve 34c represents a simulation result when the incident angle θ of FIG. 4 is 15 degrees. As shown in FIG. 8, the curves 31c, 32c, 33c, 34c are substantially consistent with each other. If the target value of the optical signal-to-noise ratio (OSNR) is set to be greater than or equal to 20 dB, the selectable refractive index of the corresponding transparent encapsulant 14 may be in a range of about 1.50 to about 1.56. Thus, a ratio of the refractive index of the transparent encapsulant 14 to the refractive index of the substrate 10 may be in a range of about 0.98 to about 1.02.

In addition, in the case 42 of FIG. 8, the substrate 10 is predetermined to be an ideal substrate having a refractive index of 1.76. The curve 31d represents a simulation result when the incident angle θ of FIG. 4 is 60 degrees. The curve 32d represents a simulation result when the incident angle θ of FIG. 4 is 45 degrees. The curve 33d represents a simulation result when the incident angle θ of FIG. 4 is 30 degrees. The curve 34d represents a simulation result when the incident angle θ of FIG. 4 is 15 degrees. As shown in FIG. 8, the curves 31d, 32d, 33d, 34d are substantially consistent with each other. If the target value of the optical signal-to-noise ratio (OSNR) is set to be greater than or equal to 20 dB, the selectable refractive index of the corresponding transparent encapsulant 14 may be in a range of about 1.725 to about 1.795. Thus, a ratio of the refractive index of the transparent encapsulant 14 to the refractive index of the substrate 10 may be in a range of about 0.98 to about 1.02.

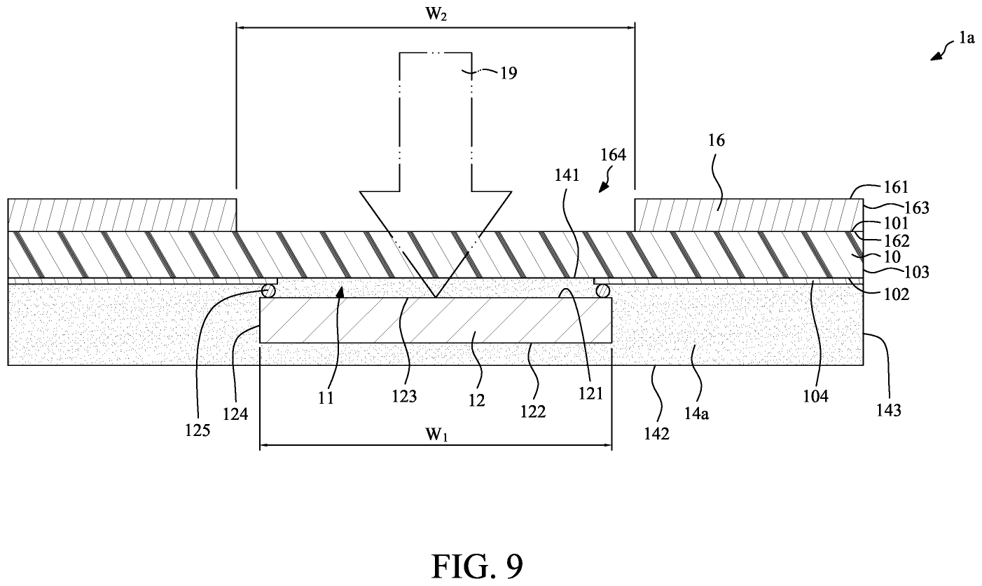

FIG. 9 illustrates a cross-sectional view of an optical sensor package structure 1a according to some embodiments of the present disclosure. The optical sensor package structure 1a of FIG. 9 is similar to the optical sensor package structure 1 of FIG. 1 to FIG. 4, except for a size of the transparent encapsulant 14a. As shown in FIG. 9, the lateral side surfaces 143 of the transparent encapsulant 14a are substantially coplanar with the lateral side surfaces 103 of the substrate 10.

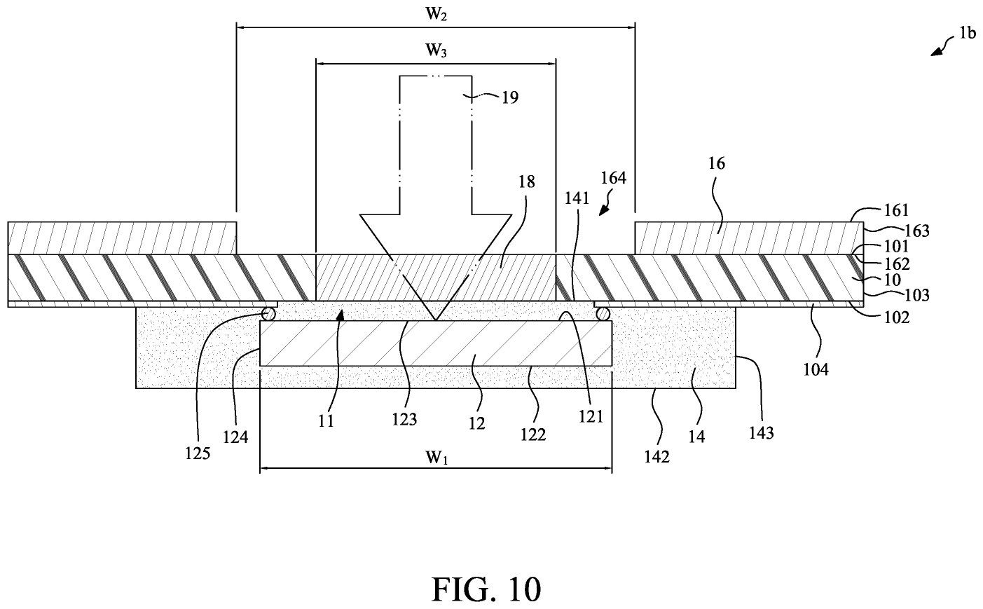

FIG. 10 illustrates a cross-sectional view of an optical sensor package structure 1b according to some embodiments of the present disclosure. The optical sensor package structure 1b of FIG. 10 is similar to the optical sensor package structure 1 of FIG. 1 to FIG. 4, except that the optical sensor package structure 1b may further include a convergence lens 18. The convergence lens 18 is disposed in the substrate 10 and corresponds to the sensor device 12 and the opening 164 of the mask layer 16. In some embodiments, the convergence lens 18 may extend through the substrate 10. Thus, a thickness of the convergence lens 18 may be substantially equal to a thickness of the substrate 10. In the present embodiment, the substrate 10 may be opaque. As shown in FIG. 10, a size (e.g., a width W3) of the convergence lens 18 may be less than the size (e.g., a width W2) of the opening 164 of the mask layer 16 and the size (e.g., a width W0 of the sensor device 12.

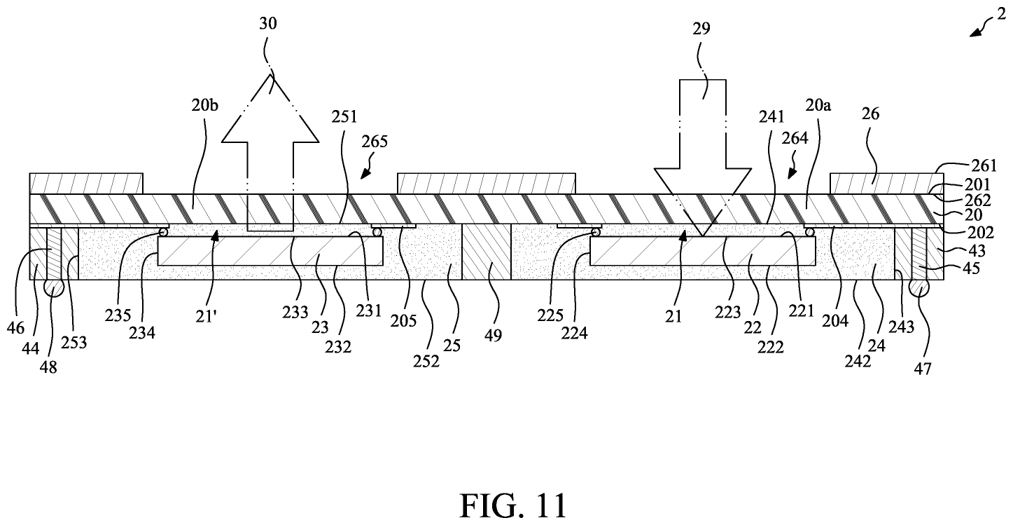

FIG. 11 illustrates a cross-sectional view of an optical module structure 2 according to some embodiments of the present disclosure. The optical module structure 2 may include a substrate 20, a light transmitter 23, a light receiver 22, a first encapsulant 24, a second encapsulant 25, a mask layer 26, a central block structure 49, a first periphery block structure 43, a second periphery block structure 44, a first conductive via 45, a second conductive via 46, a first external connector 47 and a second external connector 48.

The substrate 20 of the optical module structure 2 may be similar to or same as the substrate 10 of the optical sensor package structure 1 of FIG. 1 to FIG. 3, and may be transparent. The substrate 20 may have a first surface 201 (e.g., a top surface) and a second surface 202 (e.g., a bottom surface) opposite to the first surface 201. In some embodiments, the substrate 20 may include a first portion 20a corresponding to the light receiver 22, and a second portion 20b corresponding to the light transmitter 23. In some embodiments, the substrate 20 may further include a first circuit layer 204 and a second circuit layer 205 disposed adjacent to or disposed on the second surface 202 of the substrate 20. The first circuit layer 204 and the second circuit layer 205 may be or may be not electrically connected to each other.

The light receiver 22 of the optical module structure 2 may be similar to or same as the sensor device 12 of the optical sensor package structure 1 of FIG. 1 to FIG. 3. The light receiver 22 may be attached to and electrically connected to a first portion 20a of the substrate 20, and may have a first surface 221 (e.g., an active surface), a second surface 222 (e.g., a backside surface) opposite to the first surface 221, and four lateral side surfaces 224 extending between the first surface 221 and the second surface 222. In addition, the light receiver 22 may further have a sensing area 223 disposed adjacent to the first surface 221. The light receiver 22 may include a sensing circuit disposed in the sensing area 223 for sensing or detecting an optical signal 29 (e.g., a light). As shown in FIG. 11, the light receiver 22 is electrically connected to the substrate 20 through a flip-chip bonding. That is, the first surface 221 of the light receiver 22 is electrically connected to the first circuit layer 204 of the substrate 20 through a plurality of bumps 225. Thus, the sensing area 223 of the light receiver 22 faces the substrate 20, and a gap 21 or a space is formed between the first surface 221 (or the sensing area 223) of the light receiver 22 and the second surface 202 of the substrate 20.

The first encapsulant 24 of the optical module structure 2 may be similar to or same as the first encapsulant 14 of the optical sensor package structure 1 of FIG. 1 to FIG. 3. The first encapsulant 24 may be disposed on the second surface 202 of the substrate 20 to cover the light receiver 22 and the first portion 20a of the substrate 20. As shown in FIG. 11, the first encapsulant 24 may have a first surface 241 (e.g., a top surface), a second surface 242 (e.g., a bottom surface) opposite to the first surface 241, and four lateral side surfaces 243 extending between the first surface 241 and the second surface 242. The first surface 241 of the first encapsulant 24 may contact the second surface 202 of the substrate 20. For example, the first encapsulant 24 may include an optical molding compound such as epoxy resin with or without fillers. In some embodiments, a transparent material of the first encapsulant 24 has a light transmission of at least about 60%, at least about 70%, or at least about 80% for a wavelength in the visible range. In addition, a ratio of a refractive index of the first encapsulant 24 to a refractive index of the substrate 20 may be in a range of about 0.98 to about 1.02. As shown in FIG. 11, a portion of the first encapsulant 24 fills the gap 21 between the light receiver 22 and the substrate 20. Thus, the first encapsulant 24 may cover the sensing area 223 of the light receiver 22.

The light transmitter 23 may be attached to and electrically connected to a second portion 20b of the substrate 20, and may have a first surface 231 (e.g., an active surface), a second surface 232 (e.g., a backside surface) opposite to the first surface 231, and four lateral side surfaces 234 extending between the first surface 231 and the second surface 232. In addition, the light transmitter 23 may further have an emitting area 233 disposed adjacent to the first surface 231 for emitting an optical signal 30 (e.g., a light). For example, the light transmitter 23 may be a light emitter such as a light emitting diode (LED) or another illuminating device. As shown in FIG. 11, the light transmitter 23 is electrically connected to the substrate 20 through a flip-chip bonding. That is, the first surface 231 of the light transmitter 23 is electrically connected to the second circuit layer 205 of the substrate 20 through a plurality of bumps 235. Thus, the emitting area 233 of the light transmitter 23 faces the substrate 20, and a gap 21′ or a space is formed between the first surface 231 of the light transmitter 23 and the second surface 202 of the substrate 20.

The second encapsulant 25 may be similar to or same as the first encapsulant 24. The second encapsulant 25 may be disposed on the second surface 202 of the substrate 20 to cover the light transmitter 23 and the second portion 20b of the substrate 20. As shown in FIG. 11, the second encapsulant 25 may have a first surface 251 (e.g., a top surface), a second surface 252 (e.g., a bottom surface) opposite to the first surface 251, and four lateral side surfaces 253 extending between the first surface 251 and the second surface 252. The first surface 251 of the second encapsulant 25 may contact the second surface 202 of the substrate 20. For example, the second encapsulant 25 may include an optical molding compound such as epoxy resin with or without fillers. In some embodiments, a transparent material of the second encapsulant 25 has a light transmission of at least about 60%, at least about 70%, or at least about 80% for a wavelength in the visible range. In addition, a ratio of a refractive index of the second encapsulant 25 to a refractive index of the substrate 20 may be in a range of about 0.98 to about 1.02. As shown in FIG. 11, a portion of the second encapsulant 25 fills the gap 21′ between the light transmitter 23 and the substrate 20. Thus, the second encapsulant 25 may cover the emitting area 233.

The mask layer 26 of the optical module structure 2 may be similar to or same as the mask layer 16 of the optical sensor package structure 1 of FIG. 1 to FIG. 3. The mask layer 26 may be disposed on the first surface 201 of the substrate 20 opposite to the light transmitter 23 and the light receiver 22. The mask layer 26 may have a first surface 261 (e.g., a top surface), a second surface 262 (e.g., a bottom surface) opposite to the first surface 261, and four lateral side surfaces 263 extending between the first surface 261 and the second surface 262. The first surface 261 of the mask layer 26 may contact the first surface 201 of the substrate 20. For example, the mask layer 26 may be an opaque light-block material. In some embodiments, the material of the mask layer 26 has a light transmission of less than about 10%, less than about 5%, less than about 1%, or less than about 0.1% for a wavelength in the visible range. In addition, the mask layer 26 may define a first opening 264 corresponding to the light receiver 22 and a second opening 265 corresponding to the light transmitter 23. A size of the first opening 264 may be greater than a size of the light receiver 22.

The central block structure 49 may be disposed on the substrate 20 and between the light transmitter 23 and the light receiver 22 so as to prevent a cross-talk or an interference between the light transmitter 23 and the light receiver 22. A material of the central block structure 49 may be metal material or dielectric material (such as polyimide (PI), benzocyclobutene (BCB), dry film, FR-4 or another suitable material). The first periphery block structure 43 and the second periphery block structure 44 may be disposed on the substrate 20 and at the periphery portion of the optical module structure 2. The first periphery block structure 43 corresponds to the light receiver 22, and the second periphery block structure 44 corresponds to the light transmitter 23. A material of the first periphery block structure 43 and the second periphery block structure 44 may be dielectric material (such as polyimide (PI), benzocyclobutene (BCB), dry film, FR-4 or another suitable material). The first conductive via 45 may extend through the first periphery block structure 43 to contact the first circuit layer 204. The second conductive via 46 may extend through the second periphery block structure 44 to contact the second circuit layer 205. The first external connector 47 may be disposed on a tip of the first conductive via 45 for external connection. The second external connector 48 may be disposed on a tip of the second conductive via 46 for external connection.

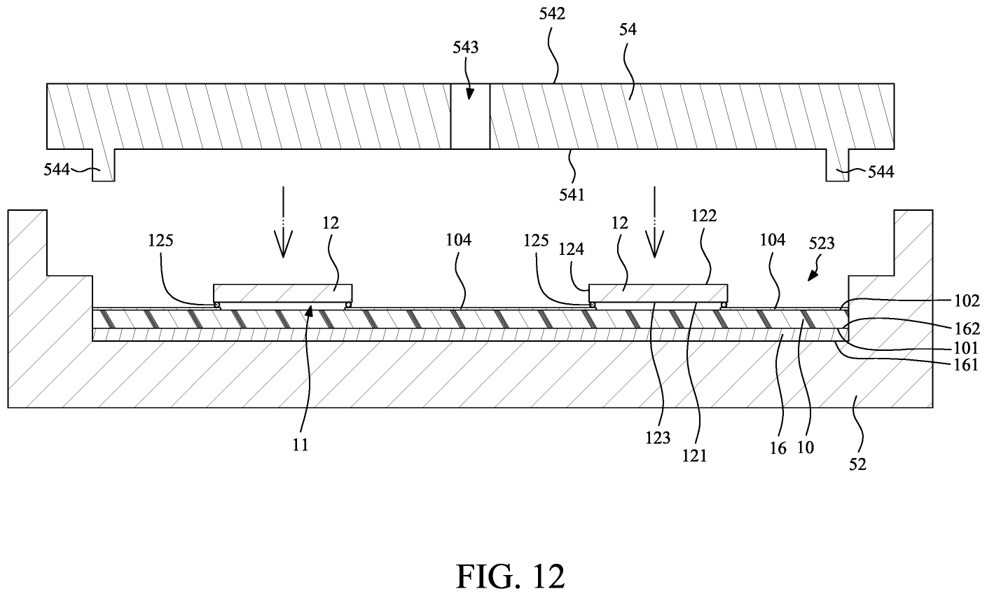

FIG. 12 through FIG. 15 illustrate a method for manufacturing an optical sensor package structure according to some embodiments of the present disclosure. In some embodiments, the method is for manufacturing the optical sensor package structure 1a shown in FIG. 9.

Referring to FIG. 12, a lower mold 52 and an upper mold 54 are provided. The lower mold 52 defines a mold cavity 523. The upper mold 54 has a first surface 541 and a second surface 542 opposite to the first surface 541, and includes at least one protrusion portion 544 protruding from the first surface 541 downward. In addition, the upper mold 54 may define an inlet hole 543 extending through the upper mold 54. In some embodiments, a material of the upper mold 54 may be glass, and a material of the lower mold 52 may be steel.

Then, a substrate 10 with a mask layer 16 are disposed in the mold cavity 523 of the lower mold 52. A first surface 161 of the mask layer 16 may contact a receiving surface of the lower mold 52. A circuit layer 104 that is disposed on the second surface 102 the substrate 10 faces upward or toward the upper mold 54. The substrate 10 may be transparent, and a refractive index of the substrate 10 may be in a range of about 1.46 to about 1.85.

Then, a plurality of sensor devices 12 may be electrically connected to the substrate 10 through a flip-chip bonding. A sensing area 123 on a first surface 121 (e.g., an active surface) of each of the sensor devices 12 faces the substrate 10, thus, a gap 11 or a space is formed between the first surface 121 (or the sensing area 123) of the sensor device 12 and the second surface 102 of the substrate 10.

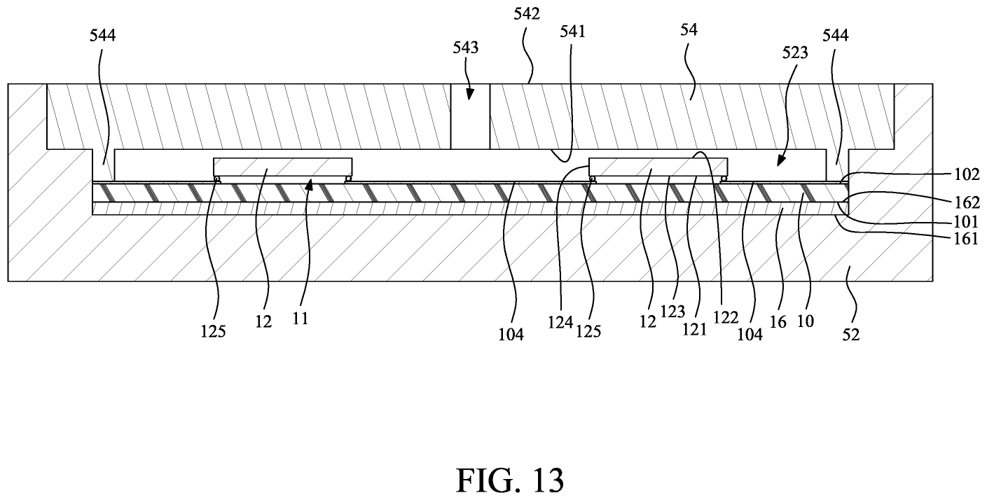

Referring to FIG. 13, the upper mold 54 is moved downward to cover and contact the lower mold 52. The upper mold 54 may be clamped with the lower mold 52 such that the mold cavity 523 becomes a substantially enclosed space. The inlet hole 543 of the upper mold 54 is in communication with the enclosed mold cavity 523. In some embodiments, the protrusion portion 544 of the upper mold 54 may contact the substrate 10.

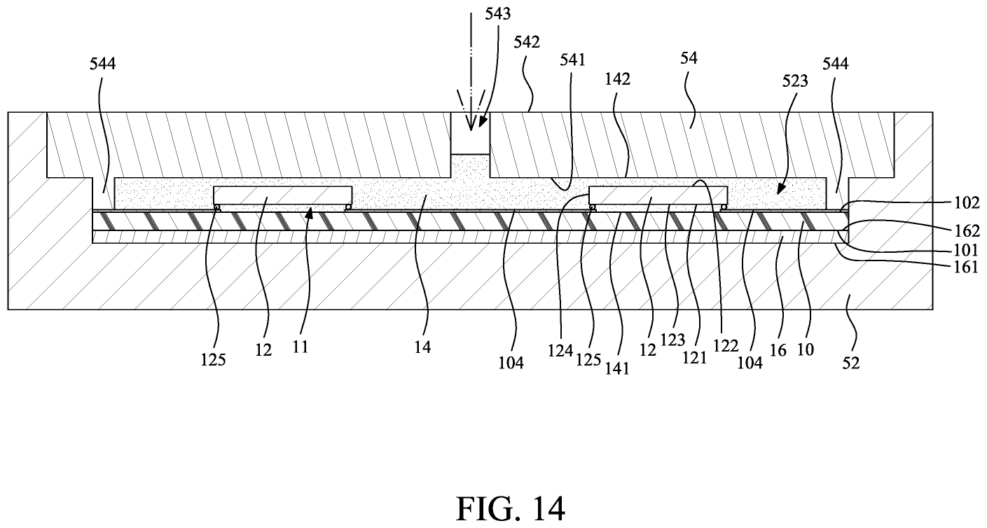

Referring to FIG. 14, a transparent encapsulant 14 may be injected into the mold cavity 523 through the inlet hole 543 by screwing, pultrusion or air pump. Thus, transparent encapsulant 14 may cover the sensor devices 12 and the second surface 102 of the substrate 10. A ratio of a refractive index of the transparent encapsulant 14 to a refractive index of the substrate 10 may be in a range of about 0.98 to about 1.02. As shown in FIG. 14, a portion of the transparent encapsulant 14 fills the gaps 11 between the sensor devices 12 and the substrate 10. In addition, the transparent encapsulant 14 may further cover the bumps 125 and a portion of the circuit layer 104 of the substrate 10.

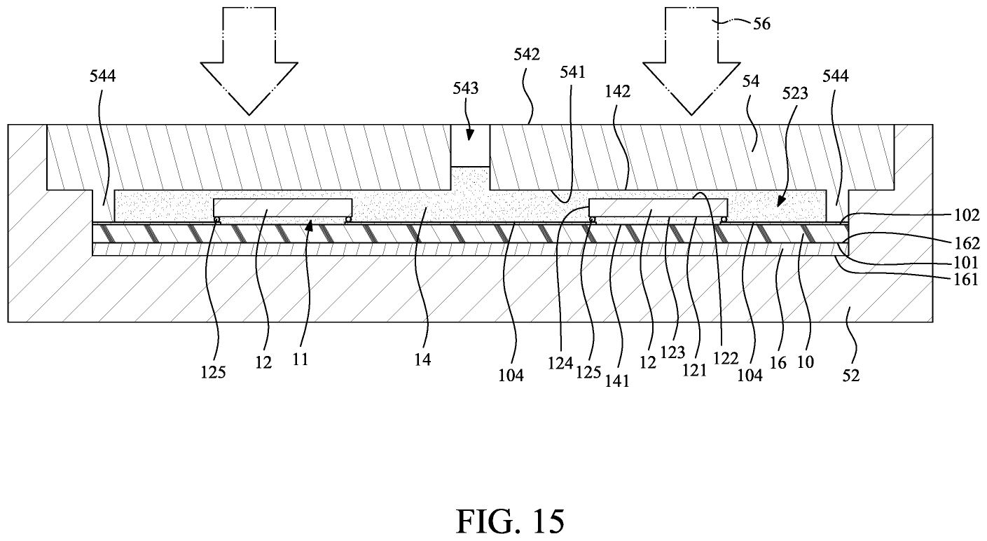

Referring to FIG. 15, a curing light 56 (e.g., UV light) is applied to the transparent encapsulant 14 through the upper mold 54, so that the transparent encapsulant 14 is exposed and cured. Then, the lower mold 52 and the upper mold 54 are removed. Then, a plurality of openings 164 are formed to extend through the mask layer 16. Each of the openings 164 corresponds to each of the sensor devices 12.

Then, a singulation process may be conducted to obtain a plurality of optical sensor package structures 1a shown in FIG. 9.

Spatial descriptions, such as “above,” “below,” “up,” “left,” “right,” “down,” “top,” “bottom,” “vertical,” “horizontal,” “side,” “higher,” “lower,” “upper,” “over,” “under,” and so forth, are indicated with respect to the orientation shown in the figures unless otherwise specified. It should be understood that the spatial descriptions used herein are for purposes of illustration only, and that practical implementations of the structures described herein can be spatially arranged in any orientation or manner, provided that the merits of embodiments of this disclosure are not deviated from by such an arrangement.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same or equal if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Two surfaces can be deemed to be coplanar or substantially coplanar if a displacement between the two surfaces is no greater than 5 μm, no greater than 2 μm, no greater than 1 μm, or no greater than 0.5 μm.

As used herein, the singular terms “a,” “an,” and “the” may include plural referents unless the context clearly dictates otherwise.

As used herein, the terms “conductive,” “electrically conductive” and “electrical conductivity” refer to an ability to transport an electric current. Electrically conductive materials typically indicate those materials that exhibit little or no opposition to the flow of an electric current. One measure of electrical conductivity is Siemens per meter (S/m). Typically, an electrically conductive material is one having a conductivity greater than approximately 104 S/m, such as at least 105 S/m or at least 106 S/m. The electrical conductivity of a material can sometimes vary with temperature. Unless otherwise specified, the electrical conductivity of a material is measured at room temperature.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other embodiments of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. An optical sensor package structure, comprising:
a substrate;
a sensor device electrically connected to the substrate, and having a sensing area facing the substrate; and
a transparent encapsulant covering the sensing area of the sensor device.

2. The optical sensor package structure of claim 1, wherein the substrate is transparent.

3. The optical sensor package structure of claim 1, wherein the sensor device has a first surface, a second surface opposite to the first surface and four lateral side surfaces extending between the first surface and the second surface the transparent encapsulant further covers a surface of the substrate and the first surface, the second surface and the four lateral side surfaces of the sensor device.

4. The optical sensor package structure of claim 1, wherein the transparent encapsulant has a light transmission of at least about 60% for a wavelength in a visible range.

5. The optical sensor package structure of claim 1, wherein a ratio of a refractive index of the transparent encapsulant to a refractive index of the substrate is in a range of 0.98 to 1.02.

6. The optical sensor package structure of claim 1, wherein a refractive index of the substrate is in a range of 1.46 to 1.85.

7. The optical sensor package structure of claim 1, further comprising a mask layer disposed on a surface of the substrate opposite to the sensor device, wherein the mask layer defines an opening corresponding to the sensor device, and a size of the opening is greater than a size of the sensor device.

8. The optical sensor package structure of claim 1, further comprising a convergence lens disposed in the substrate and corresponding to the sensor device.

9. The optical sensor package structure of claim 8, wherein the convergence lens extends through the substrate.

10. An optical sensor package structure 1, comprising:
a transparent substrate;
a sensor device electrically connected to the transparent substrate, and having a sensing area facing the transparent substrate; and
a transparent encapsulant covering the sensor device and a surface of the transparent substrate, wherein a ratio of a refractive index of the transparent encapsulant to a refractive index of the transparent substrate is in a range of 0.98 to 1.02.

11. The optical sensor package structure of claim 10, wherein a refractive index of the transparent substrate is in a range of 1.46 to 1.85.

12. The optical sensor package structure of claim 10, wherein the sensor device is electrically connected to the transparent substrate through a flip-chip bonding.

13. The optical sensor package structure of claim 10, wherein a portion of the transparent encapsulant fills a gap between the sensor device and the transparent substrate.

14. The optical sensor package structure of claim 10, wherein an optical signal-to-noise ratio (OSNR) of an optical signal received by the sensor device is greater than 20 db.

15. The optical sensor package structure of claim 10, wherein the transparent encapsulant has a light transmission of at least about 60% for a wavelength in a visible range.

16. An optical module structure, comprising:
a substrate;
a light transmitter attached to the substrate;
a light receiver attached to the substrate and having a sensing area; and
a first encapsulant covering the light receiver and a first portion of the substrate, wherein the first encapsulant is transparent and covers the sensing area of the light receiver.

17. The optical module structure of claim 16, further comprising a second encapsulant covering the light transmitter and a second portion of the substrate.

18. The optical module structure of claim 16, wherein a ratio of a refractive index of the first encapsulant to a refractive index of the substrate is in a range of 0.98 to 1.02.

19. The optical module structure of claim 16, wherein a refractive index of the substrate is in a range of 1.46 to 1.85.

20. The optical module structure of claim 16, further comprising a mask layer disposed on a surface of the substrate opposite to the light transmitter and the light receiver, wherein the mask layer defines an opening corresponding to the light receiver, and a size of the opening is greater than a size of the light receiver.
